(19) United States
(12) Patent Application Publication
(10) Pub. No.: US 2012/0156257 A1
(43) Pub. Date: Jun. 21, 2012

(54) METHOD FOR THE PREPARATION OF NANOPARTICLES

(75) Inventors: Alexandre Drogoz, Bourgoin-Jallieu (FR); Alain Constancis, Lyon (FR)

(73) Assignee: Flamel Technologies, Venissieux (FR)

(21) Appl. No.: 13/329,999

(22) Filed: Dec. 19, 2011

Related U.S. Application Data
(60) Provisional application No. 61/424,282, filed on Dec. 17, 2010.

(30) Foreign Application Priority Data
Dec. 17, 2010 (FR) FR 10 60685
Dec. 16, 2011 (IB) PCT/IB2011/055727

(51) Int. Cl. A61K 9/00; B82Y 40/00; B82Y 5/00

(52) U.S. Cl. 424/400; 977/773; 977/840

(57) ABSTRACT

The present invention relates to a novel method for the preparation of nanoparticles with a diameter smaller than or equal to 500 nm, comprising bringing a solution (1) comprising nanoparticles of a first polyelectrolyte in the charged state, bearing hydrophobic side groups, together with (2) at least one second polyelectrolyte of opposite polarity to that of the first polyelectrolyte, characterized in that the ratio Z of the number of cationic groups relative to the number of anionic groups in the mixture of the two polyelectrolytes is comprised between 0.1 and 0.75 or between 1.3 and 2; and the total mass concentration C of polyelectrolytes is strictly less than 2 mg/g of the mixture.

The invention relates to a novel method for the preparation of nanoparticles from the specific mixture of two polyelectrolytes of opposite polarity, where appropriate combined with an active ingredient.

The formulations of active ingredient must comply with a certain number of tolerance criteria, have a sufficient concentration of active ingredient, while having a low viscosity in order to allow easy injection through a needle with a small diameter, for example a 27- to 31-gauge needle.

In this field, the applicant company has succeeded in developing, as presented in document WO 2008/135561, stable suspensions with low viscosity, constituted by microparticles loaded with active ingredient. These microparticles, capable of releasing the active ingredient over an extended period, are more particularly formed from the mixture, under specific conditions, of two polyelectrolyte polymers (PE1) and (PE2) of opposite polarity, at least one of which bears hydrophobic groups. This mixture leads to microparticles of a size comprised between 1 and 100 μm.

However, the formulations of microparticles are not suitable for intravenous administration and may, within the framework of administration by subcutaneous route, pose problems of intolerance.

Consequently, from the viewpoint of administration of active ingredients by parenteral, in particular intravenous or subcutaneous, route, it would be preferable to have suspensions of particles of even smaller size, and in particular on the nanometric scale.

Therefore there is still a need for a method for the preparation of stable suspensions of nanoparticles of active ingredient, particularly suitable for an administration by parenteral, in particular intravenous, route.

The present invention specifically aims to propose a novel method allowing such suspensions of nanoparticles to be obtained.

Against all expectations, the inventors have discovered that it is possible to obtain fluid formulations of nanoparticles, where appropriate loaded with active ingredient, from a particular mixture of specific polyelectrolytes.

More precisely, according to a first of its aspects, the present invention relates to a method for the preparation of nanoparticles with an average diameter less than or equal to 500 nm, comprising at least the stages consisting of:

(1) having an aqueous solution comprising nanoparticles of a first polyelectrolyte in the charged state bearing hydrophobic side groups;

(2) bringing said solution (1) together with at least one second polyelectrolyte of opposite polarity to that of the first polyelectrolyte, with the cationic polyelectrolyte being added to a solution of anionic polyelectrolyte to form a mixture with an excess of anionic charge; or the anionic polyelectrolyte being added to a solution of cationic polyelectrolyte to form a mixture with an excess of cationic charge; and

(3) having the nanoparticles thus formed; with:

said anionic and cationic polyelectrolytes having a linear backbone of the polyamino acid type, devoid of side groups of the polyalkylene glycol type, and having a degree of polymerization less than or equal to 2,000;

the molar ratio Z of the number of cationic groups relative to the number of anionic groups in the mixture of the two polyelectrolytes being comprised between 0.1 and 0.75 or between 1.3 and 2; and

the total mass concentration C of polyelectrolytes being strictly less than 2 mg/g of said mixture.

According to a particular embodiment, the method of the invention comprises, after stage (3), one or more stages of concentration (4), in particular by tangential or frontal ultrafiltration, centrifugation, evaporation or lyophilization.

According to an embodiment variant of the method of the invention, where Z is comprised between 0.1 and 0.75, in other words when the final mixture of the polyelectrolytes has an excess of anionic charge, stage (1) consists of preparing an aqueous solution of nanoparticles of an anionic polyelectrolyte. Stage (2) then consists of adding the cationic polyelectrolyte, in particular in the form of an aqueous solution, to the solution of the first polyelectrolyte preferably placed under moderate stirring.

Inversely, where Z is comprised between 1.3 and 2, in other words when the final mixture of the polyelectrolytes has an excess of cationic charge, stage (1) consists of preparing an aqueous solution of nanoparticles of a cationic polyelectrolyte. Stage (2) then consists of adding the anionic polyelectrolyte, in particular in the form of an aqueous solution, to the solution of the first polyelectrolyte preferably placed under moderate stirring.

The method of the invention is particularly advantageous, with regard to the characteristics of its stage (2), for preventing the formation of particles not according to the invention, i.e. of average diameter strictly greater than 500 nm.

According to a particularly advantageous embodiment, the nanoparticles of the aqueous solution of stage (1) are non-covalently combined with an active ingredient.

Such an aqueous solution of nanoparticles of active ingredient is obtained by adding the active ingredient to an aqueous colloidal solution of the first polyelectrolyte, said active ingredient combining non-covalently with the nanoparticles of the first polyelectrolyte.

The formulations of nanoparticles obtained at the end of the method of the invention prove to be advantageous in several respects.

Firstly, the nanometric size of the particles obtained by the method of the invention is particularly suited to administration of the formulation of active ingredients by intravenous or subcutaneous route. The present invention thus proves to be particularly advantageous with regard to the parenteral administration of active ingredients used for the treatment of cancers.

Moreover, the polyelectrolytes used in the method of the invention are biocompatible. They are perfectly tolerated and degrade rapidly, i.e. on a time scale of a few days to a few weeks.

The nanoparticles obtained by the method of the invention, combined with active ingredients, prove to be particularly advantageous for conveying active ingredients, in particular proteinic, peptidic active ingredients and/or solubilizing active ingredients of low molecular mass.

In particular, these nanoparticles are advantageously capable of releasing the active ingredient over an extended period.

The nanoparticles loaded with active ingredient obtained according to the method of the invention advantageously have a high density. Such a density allows the release to be slowed down by steric barrier effect (matrix effect), an effect in addition to the non-covalent combination of the active ingredient with the nanoparticles of polyelectrolytes.

Moreover, a suspension of nanoparticles according to the invention advantageously has an excellent stability. The mixture obtained at the end of the method of the invention can then undergo one or more stages of concentration, in particular by tangential or frontal ultrafiltration, centrifugation, evaporation or lyophilization, without impairing the physicochemical properties of the suspension, in particular in terms of viscosity, particle size, colloidal or chemical stability. It is thus possible according to the invention to obtain a stable suspension of nanoparticles that is fluid and sufficiently concentrated.

Moreover, the suspension of nanoparticles according to the invention can be formed extemporaneously at the time of administration by simply mixing two liquid suspensions prepared as described above. Thus, these suspensions of nanoparticles can easily be stored, allowing a limited production cost on the industrial scale to be envisaged.

Finally, the active ingredient is used in an aqueous method not requiring excessive temperature, significant shearing, surfactant, or organic solvent, which advantageously makes it possible to avoid any potential degradation of the active ingredient. Such a characteristic appears to be particularly advantageous with regard to certain active ingredients, such as peptides and proteins, which can potentially be degraded when they are subjected to the abovementioned conditions.

Other characteristics, advantages and embodiments of the method according to the invention will become more apparent on reading the description which follows.

In the remainder of the text, the expressions “comprised between . . . and . . . ”, “ranging from . . . to . . . ” and “varying from . . . to . . . ” are equivalent and are meant to signify that the limits are inclusive, unless otherwise specified.

Polyelectrolytes

As previously stated, the method of the invention utilizes the mixture of at least two polyelectrolytes of opposite polarity, in other words of at least one anionic polyelectrolyte and at least one cationic polyelectrolyte.

By “polyelectrolyte” is meant, within the meaning of the present invention, a polymer bearing groups capable of ionizing in water, in particular at a pH ranging from 5 to 8, which creates a charge on the polymer. Thus, in solution in a polar solvent such as water, a polyelectrolyte dissociates, causing charges to appear on its backbone and counter-ions in solution.

The polyelectrolytes according to the invention can comprise a set of identical or different electrolyte groups.

Unless otherwise specified, the polyelectrolytes are described, throughout the remainder of the description, as they appear at the pH value of a mixture of the anionic and cationic polyelectrolytes during stage (2) of the method of the invention. The description of a group as “cationic” or as “anionic” is considered for example in the light of the charge borne by this group at this pH value of a mixture of the anionic and cationic polyelectrolytes. Similarly, the polarity of a polyelectrolyte is defined in the light of the overall charge borne by this polyelectrolyte at this pH value.

More particularly, by “anionic polyelectrolyte” is meant a polyelectrolyte having a negative overall charge at the pH value of a mixture of the two polyelectrolytes.

Similarly, by “cationic polyelectrolyte” is meant a polyelectrolyte having a positive overall charge at the pH value of a mixture of the two polyelectrolytes.

By “overall charge” of a polyelectrolyte is meant the algebraic sum of all of the positive and negative charges borne by this polyelectrolyte.

Preferably, the pH value of a mixture of the anionic and cationic polyelectrolytes leading to the formation of the nanoparticles ranges from 5 to 8, preferably from 6 to 7.5.

In particular, according to a particularly preferred embodiment, the aqueous solution (1) has a pH value ranging from 5 to 8, in particular from 6 to 7.5 and more particularly of approximately 7.

According to a particular embodiment, stage (2) of the method of the invention comprises at least:
the preparation of an aqueous solution of the second polyelectrolyte, in particular with a pH value ranging from 5 to 8, and advantageously with a pH value identical to that of the aqueous solution (1); and
the mixing of said aqueous solution of the second polyelectrolyte with said aqueous solution (1).

According to one of the aspects of the invention, the first polyelectrolyte bears hydrophobic side groups. This polyelectrolyte is in particular capable of spontaneously forming nanoparticles when it is dispersed in an aqueous medium with a pH ranging from 5 to 8, in particular water.

Without wishing to be bound by the theory, it is possible to suggest that the supramolecular combination of the hydrophobic groups to form hydrophobic domains leads to the formation of nanoparticles. Each nanoparticle is thus constituted by one or more polyelectrolyte chains more or less condensed around these hydrophobic domains.

Preferably, the nanoparticles formed by the first polyelectrolyte, bearing hydrophobic side groups, have an average diameter ranging from 10 to 100 nm, in particular from 10 to 70 nm, and more particularly ranging from 10 to 50 nm.

According to another particular embodiment, the second polyelectrolyte of stage (2) of the method of the invention also bears hydrophobic groups. It can also be capable of forming nanoparticles when it is dispersed in an aqueous medium with a pH ranging from 5 to 8, in particular water.

Linear Backbone of the Polyamino Acid Type

As mentioned previously, the polyelectrolytes considered according to the invention have a linear backbone of the polyamino acid type, i.e. comprising amino acid residues.

Advantageously, the polyelectrolytes according to the invention are biodegradable.

Within the meaning of the invention, the term “polyamino acid” covers both natural polyamino acids and synthetic polyamino acids.

The polyamino acids are linear polymers, advantageously composed of alpha-amino acids linked by peptide bonds.

There are numerous synthesis techniques for forming block or random copolymers, multiple-chain polymers and polymers containing a particular sequence of amino acids (cf. Encyclopedia of Polymer Science and Engineering, volume 12, page 786; John Wiley & Sons).

A person skilled in the art is capable, by virtue of their knowledge, of implementing these techniques in order to obtain polymers suitable for the invention. In particular, reference can also be made to the teaching of documents WO 96/29991, WO 03/104303, WO 2006/079614 and WO 2008/135563.

According to a preferred embodiment variant, the polyamino acid chain is constituted by a homopolymer of alpha-L-glutamate or of alpha-L-glutamic acid.

According to another embodiment variant, the polyamino acid chain is constituted by a homopolymer of alpha-L-aspartate or of alpha-L-aspartic acid.

According to another embodiment variant, the polyamino acid chain is constituted by a copolymer of alpha-L-aspartate/alpha-L-glutamate or of alpha-L-aspartic/alpha-L-glutamic acid.

Such polyamino acids are in particular described in documents WO 03/104303, WO 2006/079614 and WO 2008/135563, the contents of which are incorporated by way of reference. These polyamino acids can also be of the type of those described in Patent Application WO 00/30618.

These polymers can be obtained by methods known to a person skilled in the art.

A certain number of polymers which can be used according to the invention, for example of the poly(alpha-L-glutamic acid), poly(alpha-D-glutamic acid), poly(alpha-D,L-glutamate) and poly(gamma-L-glutamic acid) type of variable masses are commercially available.

Poly(L-glutamic acid) can also be synthesized according to the route described in Patent Application FR 2 801 226.

According to a particularly advantageous embodiment, the anionic polyelectrolyte considered according to the invention is of the following formula (I) or one of its pharmaceutically acceptable salts,

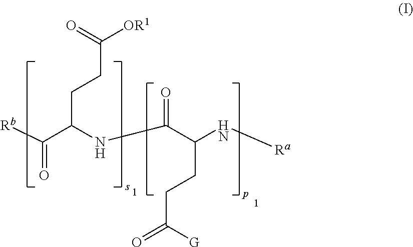

in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
   R⁵ represents a hydrogen atom, a linear C₁ to C₁₀ alkyl group, a branched C₃ to C₁₀ alkyl group, or a benzyl group;
   R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-, preferably alpha-tocopheryl-;
   s₁ corresponds to the average number of non-grafted glutamate monomers, anionic at neutral pH,
   p₁ corresponds to the average number of glutamate monomers bearing a hydrophobic group G,
p₁ optionally being zero,
   the degree of polymerization DP₁=(s₁+p₁) is less than or equal to 2,000, in particular less than 700, more particularly ranging from 40 to 450, in particular from 40 to 250, and in particular from 40 to 150,
the chain formation of the monomers of said general formula (I) can be random, monoblock or multiblock type.

According to a particularly preferred embodiment of the invention, the anionic polyelectrolyte of formula (I) has a mole fraction x_{P1} of monomers bearing hydrophobic groups such that x_{P1}=+p₁/(s₁+p₁) varies from 2 to 22%, in particular from 4 to 12%.

According to a particularly advantageous embodiment, the cationic polyelectrolyte according to the invention is of the following formula (II) or one of its pharmaceutically acceptable salts,

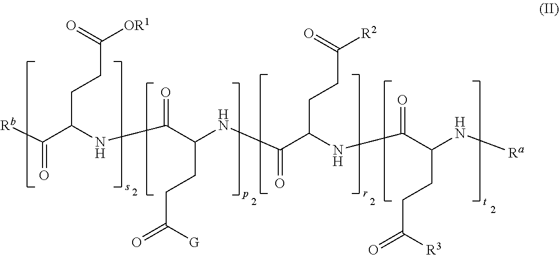

in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
   R⁵ represents a hydrogen atom, a linear C₁ to C₁₀ alkyl group, a branched C₃ to C₁₀ alkyl group, or a benzyl group;
   R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-, preferably alpha-tocopheryl-;
R² represents a cationic group, in particular arginine;
R³ represents a neutral group chosen from: hydroxyethylamino-, dihydroxypropylamino-;
   s₂ corresponds to the average number of non-grafted glutamate monomers, anionic at neutral pH,
   p₂ corresponds to the average number of glutamate monomers bearing a hydrophobic group G,
   r₂ corresponds to the average number of glutamate monomers bearing a cationic group R², and
   t₂ corresponds to the average number of glutamate monomers bearing a neutral group R³,

s₂, p₂ and t₂ optionally being zero, and
the degree of polymerization DP₂=(s₂+p₂+r₂+t₂) is less than or equal to 2,000, in particular less than 700, more particularly varies from 40 to 450, in particular from 40 to 250, and in particular from 40 to 150,
the chain formation of the monomers of said general formula (II) can be random, monoblock or multiblock type.

Of course, the cationic polyelectrolyte corresponding to formula (II) is such that the overall charge of the polyelectrolyte (r₂-s₂) is positive.

According to a particularly preferred embodiment of the invention, the cationic polyelectrolyte of formula (II) has a mole fraction Xp₂ of monomers bearing hydrophobic groups such that x_{P2}=p₂/(s₂+p₂+r₂+t₂) varies from 2 to 22%, in particular from 4 to 12%.

Of course, the nature of the anionic and cationic polyelectrolytes utilized in the method of the invention is such that at least one of the two polyelectrolytes bears hydrophobic side groups G.

According to one of the aspects of the present invention, the quantities and the nature of the anionic and cationic polyelectrolytes utilized in the method of the invention are such that the molar ratio, denoted Z, of the number of cationic groups relative to the number of anionic groups in the mixture of the two polyelectrolytes is comprised between 0.1 and 0.75 or between 1.3 and 2.

Preferably, the molar ratio Z is comprised between 0.3 and 0.75, more particularly between 0.5 and 0.75, or between 1.3 and 1.5.

The molar ratio Z can be defined, with regard to the quantities and the nature of the polyelectrolytes introduced during the preparation of the nanoparticles according to the method of the invention, by the following formula:

$$Z = \frac{( {x}_{c 2} \cdot {m}_{2} \cdot {C}_{2} \cdot {DP}_{2} / {M}_{2} )}{( {x}_{a 1} \cdot {m}_{1} \cdot {C}_{1} \cdot {DP}_{1} / {M}_{1} ) + ( {x}_{a 2} \cdot {m}_{2} \cdot {C}_{2} \cdot {DP}_{2} / {M}_{2} )}$$

in which:
m₁ and m₂ respectively represent the mass quantities of the solutions before mixing the anionic polyelectrolyte and the cationic polyelectrolyte of respective mass concentrations of polymer (before mixing) C₁ and C₂;
DP₁ and DP₂ respectively represent the degrees of polymerization of the anionic polyelectrolyte and of the cationic polyelectrolyte;
M₁ and M₂ respectively represent the molar masses of the anionic polyelectrolyte and of the cationic polyelectrolyte;
x_{c2} represents the mole fraction of monomers bearing cationic groups in the cationic polyelectrolyte;
xₐ₁ and xₐ₂ respectively represent the mole fractions of monomers bearing anionic groups of the anionic polyelectrolyte and of the cationic polyelectrolyte;

According to another aspect of the present invention, the quantities and the nature of the anionic and cationic polyelectrolytes utilized in the method of the invention are such that the total mass concentration C of polyelectrolytes is strictly less than 2 mg/g of the mixture.

In particular, the total mass concentration C of polyelectrolytes is comprised between 0.5 and 1.8 mg/g, in particular between 1 and 1.5 mg/g of the mixture.

Within the framework of utilizing the polyelectrolytes in aqueous solution, the total mass concentration C of polyelectrolytes according to the invention is strictly less than 2 mg/g of the aqueous solution obtained at the end of stage (2) of the method of the invention.

The total mass concentration C of polyelectrolytes can be defined by:

C=(m₁·C₁+m₂·C₂)/(m₁+m₂),

with m₁, m₂, C₁ and C₂ as previously defined.

According to a first embodiment, the anionic and cationic polyelectrolytes are such that:
the degree of polymerization of the anionic and cationic polyelectrolytes is comprised between 50 and 220;
the anionic polyelectrolyte bears 4 to 12 molar % of hydrophobic side groups, distributed randomly.

According to a second embodiment, the anionic and cationic polyelectrolytes are such that:
the degree of polymerization of the anionic and cationic polyelectrolytes is comprised between 50 and 220;
t₂ is zero, i.e. the cationic polyelectrolyte is devoid of neutral groups;
the cationic polyelectrolyte and the anionic polyelectrolyte both bear 4 to 12 molar % of hydrophobic side groups, distributed randomly.

According to a third embodiment, the anionic and cationic polyelectrolytes are such that:
the degree of polymerization of the anionic and cationic polyelectrolytes is comprised between 50 and 220;
the anionic polyelectrolyte bears 4 to 12 molar % of hydrophobic side groups, distributed randomly; and
the cationic polyelectrolyte bears 30 to 60 molar % of cationic side groups, in particular arginine.

According to a fourth embodiment, the anionic and cationic polyelectrolytes are such that:
the degree of polymerization of the anionic and cationic polyelectrolytes is comprised between 50 and 220;
the anionic polyelectrolyte bears 18 to 22 molar % of hydrophobic side groups, distributed randomly.

### Nanoparticles

As previously stated, the nanoparticles formed according to the invention have an average diameter less than or equal to 500 nm.

Preferably, the size of the nanoparticles can vary from 20 to 300 nm, in particular from 50 to 200 nm.

The size of the nanoparticles can be measured by quasi-elastic light scattering.

Test for Measuring Particle Size by Quasi-Elastic Light Scattering

The particle size is characterized by the volume-average hydrodynamic diameter, obtained according to methods of measurement that are well known to a person skilled in the art, for example using a device of the ALV CGS-3 type.

Generally, the measurements are carried out with solutions of polymers prepared at concentrations of 1 mg/g in 0.15 M NaCl medium and stirred for 24 h. These solutions are then filtered on 0.8-0.2 μm, before being analysed by dynamic light scattering.

When using a device of the ALV CGS-3 type, operating with a vertically polarized He—Ne laser beam of wavelength 632.8 nm, the scattering angle is 140° and the signal acquisition time is 10 minutes. Measurement is repeated 3 times on two samples of solution. The result is the average of the 6 measurements.

Within the meaning of the invention, by “anionic nanoparticles” is meant nanoparticles the overall charge of which at neutral pH is negative; and by “cationic nanoparticles” is meant nanoparticles the overall charge of which at neutral pH is positive.

Active Ingredients

As mentioned previously, the method of the invention can also utilize at least one active ingredient.

The formulations of nanoparticles obtained by the method of the invention can thus be utilized for the purpose of conveying active ingredients.

According to a particularly preferred embodiment, the active ingredient is utilized in the aqueous solution of stage (1). Advantageously, the active ingredient combines non-covalently with the nanoparticles of the aqueous solution of stage (1).

The terms “combination” or “combined” used to describe the relationships between one or more active ingredients and the polyelectrolyte(s) mean that the active ingredient or ingredients are combined with the polyelectrolyte(s) by non-covalent physical interactions, in particular hydrophobic interactions, and/or electrostatic interactions and/or hydrogen bonds and/or via steric encapsulation by the polyelectrolytes.

This active ingredient can be a molecule of therapeutic, cosmetic or prophylactic interest or of interest for imaging.

It is preferably chosen from the group comprising: proteins, glycoproteins, proteins covalently bound to one or more polyalkylene glycol chains [preferably polyethylene glycol (PEG)], peptides, polysaccharides, liposaccharides, oligonucleotides, polynucleotides, synthetic pharmaceutical substances and mixtures thereof.

More preferably, the active ingredient is chosen from the subgroup comprising erythropoietins, haemoglobin raffimer, analogues or derivatives thereof; oxytocin, vasopressin, adrenocorticotropic hormone, growth factor, blood factors, haemoglobin, cytochromes, the albumins prolactin, luliberin (luteinizing hormone releasing hormone or LHRH) or analogues such as leuprolide, goserelin, triptorelin, buserelin, nafarelin; LHRH antagonists, LHRH competitors, human, porcine or bovine growth hormones (GH), growth hormone releasing hormone, insulin, somatostatin, glucagon, interleukins or mixtures thereof, interferons such as interferon alpha, alpha-2b, beta, beta-1a, or gamma; gastrin, tetragastrin, pentagastrin, urogastrone, secretin, calcitonin, enkephalins, endomorphins, angiotensins, thyrotropin-releasing factor (TRF), tumour necrosis factor (TNF), nerve growth factor (NGF), growth factors such as beclapermin, trafermin, ancestim, keratinocyte growth factor, granulocyte-colony stimulating factor (G-CSF), granulocyte-macrophage-colony stimulating factor (GM-CSF), macrophage-colony stimulating factor (M-CSF), heparinase, bone morphogenetic protein (BMP), hANP, glucagon-like peptide (GLP-I), VEG-F, recombinant hepatitis B antigen (rHBsAg), renin, cytokines, bradykinin, bacitracins, polymyxins, colistines, tyrocidine, gramicidins, etanercept, imiglucerase, drotrecogin alpha, cyclosporines and synthetic analogues, pharmaceutically active modifications and fragments of enzymes, of cytokines, of antibodies, of antigens and of vaccines, antibodies such as rituximab, infliximab, trastuzumab, adalimumab, omalizumab, tositumomab, efalizumab, and cetuximab.

Other active ingredients are polysaccharides (for example heparin) and oligo- or polynucleotides, DNA, RNA, iRNA, antibiotics and living cells, risperidone, zuclopenthixol, fluphenazine, perphenazine, flupentixol, haloperidol, fluspirilene, quetiapine, clozapine, amisulpride, sulpiride, ziprasidone; etc.

More particularly, the active ingredient is chosen from growth hormone, interferon alpha and calcitonin.

Advantageously, the suspension of nanoparticles obtained according to the method of the invention is suitable for administration by parenteral route, in particular by intravenous route.

Preferably, it has a viscosity, measured at 20° C. and at a shear rate of 10 s⁻¹, ranging from 1 to 500, preferably from 2 to 200 mPa·s.

The viscosity can be measured at 20° C., using standard equipment such as for example an imposed stress rheometer (Gemini, Bohlin) with geometry of the cone and plate type (4 cm and angle of 2°), or a Malvern Nanosizer viscometer, following the manufacturer's instructions.

According to a particular embodiment, the suspension of nanoparticles obtained at the end of stage (2) of the method according to the invention described above is subjected to one or more stages of concentration, in particular by tangential or frontal ultrafiltration, centrifugation, evaporation or lyophilization.

According to another embodiment variant, the method according to the invention can then comprise a stage of dehydrating the suspension of the obtained particles (for example by lyophilization or atomization), in order to obtain them in the form of dry powder.

Advantageously, the nanoparticles according to the invention are stable in the lyophilized form. Moreover, they are easy to redispere after lyophilization. Thus, the suspension of nanoparticles obtained according to the invention can be lyophilized then reconstituted in aqueous solution, without affecting the properties of the nanoparticles obtained.

The method of the invention can allow the preparation of novel pharmaceutical, phytosanitary, food, cosmetic or dietetic preparations made from the compositions according to the invention.

The suspension of nanoparticles obtained at the end of stage (2) of the invention can thus undergo one or more subsequent stages of conversion, in order to prepare a composition in the form of a powder, a solution, a suspension, a tablet or a gelatin capsule.

The composition obtained at the end of the method of the invention can in particular be intended for the preparation of a medicament.

It can be intended for administration by oral route or by parenteral route, in particular by parenteral route and more particularly by subcutaneous route.

The invention will be better explained by the following examples, given only by way of illustration.

### EXAMPLES

### Example 1

### Synthesis of the Anionic Polyelectrolytes PA: Polyglutamates Grafted with Vitamin E

The synthesis of such polymers is described in particular in the Applicant's International Application WO 03/104303.

Table 1 below describes the characteristics of the anionic polyelectrolytes PA (the notations p₁ and s₁ refer to formula (I) of the description; the notations xₚ₁, xₐ₁, DP₁ are those defined in the description).

TABLE 1

| | | M₁ | | |
|---|---|---|---|---|
| Characteristics | DP₁ | (g/mol) | xₚ₁ (%) | xₐ₁ (%) |
| PA₁ (p₁ = 5, s₁ = 95) | 220 | 37540 | 5 | 95 |
| PA₂ (p₁ = 5, s₁ = 95) | 100 | 17064 | 5 | 95 |
| PA₃ (p₁ = 2.5, s₁ = 47.5) | 50 | 8532 | 5 | 95 |
| PA₄ (p₁ = 10, s₁ = 90) | 100 | 19017 | 10 | 90 |
| PA₅ (p₁ = 20, s₁ = 80) | 100 | 22924 | 20 | 80 |

### Example 2

### Synthesis of the Cationic Polyelectrolytes PC

Polyglutamates Grafted with Vitamin E and Arginine (PC₁, PC₂ and PC₃)

The synthesis of these polymers is described in particular in the Applicant's International Application WO 2008/135563.

Polyglutamates Grafted with Vitamin E, Arginine and Ethanolamine (PC₄ and PC₅)

The synthesis of this polymer is similar to the synthesis of the polymers PC₁, PC₂ and PC₃ and in addition comprises a stage of grafting of ethanolamine. This grafting stage is described in the Applicant's International Application WO 2006/079614.

Table 2 below describes the characteristics of the cationic polyelectrolytes PC (the notations p₂, r₂, s₂ and t₂ refer to formula (II) of the description; the notations DP₂, M₂, xₚ₂, xₐ₂ and x_{c2} are those defined previously in the description).

TABLE 2

| | | M₂ | xₚ₂ | xₐ₂ | x_{c2} |
|---|---|---|---|---|---|
| Characteristics | DP₂ | (g/mol) | (%) | (%) | (%) |
| PC₁ (p₂ = 5, r₂ = 80, s₂ = 15, | 100 | 30640 | 5 | 15 | 80 |
| t₂ = 0) | | | | | |
| PC₂ (p₂ = 5, r₂ = 30, s₂ = 15, | 50 | 14600 | 10 | 30 | 60 |
| t₂ = 0) | | | | | |
| PC₃ (p₂ = 6, r₂ = 90, s₂ = 4, | 25 | 8182 | 6 | 4 | 90 |
| t₂ = 0) | | | | | |
| PC₄ (p₂ = 10, r₂ = 40, s₂ = 10, | 100 | 26649 | 10 | 10 | 40 |
| t₂ = 40)^{(a)} | | | | | |
| PC₅ (p₂ = 5, r₂ = 40, s₂ = 10, | 100 | 24696 | 5 | 15 | 40 |
| t₂ = 10)^{(a)} | | | | | |

^{(a)}t₂ refers in this case to neutral grafts of the hydroxyethylamino- type

### Example 3

### Preparation of Particles Based on the Two Polyelectrolytes PA and PC for Different Values of Z

The anionic polyelectrolyte PA is diluted in a 10 mM solution of NaCl in order to obtain a solution with the concentration C₁.

The cationic polyelectrolyte PC is diluted in a 10 mM solution of NaCl in order to obtain a solution with the concentration C₂.

The method then differs in the order of addition, depending on whether the final mixture sought has an excess of anionic charge or an excess of cationic charge:
for sought mixtures with an excess of anionic charge (tests e 1.1 to e 1.9 in the table below), a mass m₁ of anionic polyelectrolyte PA at concentration C₁ is placed in a beaker under moderate stirring and a mass m₂ of cationic polyelectrolyte PC at concentration C₂ is then added.
for sought mixtures with an excess of cationic charge (tests e 1.10 and e 1.11 in the table below), a mass m₂ of cationic polyelectrolyte PC at concentration C₂ is placed in a beaker under moderate stirring and a mass m₁ of the anionic polyelectrolyte PA at concentration C₁ is then added.

The diameter of the nanoparticles obtained is measured by quasi-elastic light scattering, as described previously.

The overall Zeta charge is measured by the measurement of the Zeta potential at neutral pH.

The different values of the ratio Z (cationic groups/anionic groups molar ratio), the total mass concentration C of polyelectrolytes in the mixture, the diameter, and the Zeta potential of the nanoparticles formed for different mixtures of solutions of the two polyelectrolytes PA and PC are shown in Table 3 below.

TABLE 3

| | | | | | | | | Volume | |
|---|---|---|---|---|---|---|---|---|---|
| | | m₁ | C₁ | m₂ | C₂ | C | | diameter | |
| Tests | Polymers | (g) | (mg/g) | (g) | (mg/g) | (mg/g) | Z | (nm) | Zeta (mV) |
| e 1.1 | PA₁/PC₁ | 79.97 | 0.51 | 8.90 | 8.04 | 1.26 | 0.73 | 95 | −53 |
| e 1.2 | PA₁/PC₄ | 85.00 | 0.53 | 9.67 | 8.00 | 1.29 | 0.41 | 249 | −46 |
| e 1.3 | PA₂/PC₁ | 79.95 | 0.50 | 8.52 | 8.04 | 1.23 | 0.70 | 64 | −56 |
| e 1.4 | PA₂/PC₁ | 2.40 | 1.32 | 2.70 | 2.01 | 1.69 | 0.70 | 150 | −55 |
| e 1.5 | PA₂/PC₂ | 9.00 | 0.86 | 16.95 | 1.21 | 1.09 | 0.66 | 178 | −48 |
| e 1.6 | PA₂/PC₄ | 84.95 | 0.50 | 10.78 | 8.00 | 1.34 | 0.48 | 134 | −45 |
| e 1.7 | PA₄/PC₅ | 3.00 | 1.03 | 5.40 | 1.01 | 1.02 | 0.49 | 46 | −32 |
| e 1.8 | PA₅/PC₅ | 3.01 | 1.03 | 3.30 | 1.05 | 1.04 | 0.43 | 20 | −35 |
| e 1.9 | PA₁/PC₅ | 79.04 | 0.50 | 14.34 | 8.02 | 1.65 | 0.64 | 251 | −34 |
| e 1.10 | PA₃/PC₁ | 10.08 | 0.95 | 40.05 | 1.00 | 0.98 | 1.43 | 82 | +38 |
| e 1.11 | PA₄/PC₅ | 2.01 | 1.00 | 17.20 | 1.01 | 1.01 | 1.41 | 290 | +32 |

The results show that it is possible to obtain, from the mixture of anionic PA and cationic PC polyelectrolytes according to the invention, nanoparticles of a size less than or equal to 500 nm, in accordance with the invention.

### Example 4

### Comparative

### Formulations Having a Total Polymer Concentration after Mixing Greater than 2 mg/g or Having a Charge Molar Ratio Z Strictly Greater than 0.75 and Strictly Less than 1.3

The anionic and cationic polyelectrolytes used are chosen from the previously described polyelectrolytes.

A quantity m₁ of a solution of the anionic polyelectrolyte PA described in Example 1 is added, at concentration C₁ in a 10 mM solution of NaCl, to a quantity m₂ of a solution of the cationic polyelectrolyte PC described in Example 2, diluted beforehand to a concentration C₂ in a 10 mM solution of NaCl.

TABLE 4

| | | | C₁ | | C₂ | C | | |
|---|---|---|---|---|---|---|---|---|
| | Poly- | m₁ | (mg/ | m₂ | (mg/ | (mg/ | | Volume |
| Tests | mers | (g) | g) | (g) | g) | g) | Z | diameter (nm) |
| e 2.1 | PA₂/ | 2.40 | 11.06 | 3.52 | 12.96 | 12.19 | 0.70 | Flocculation |
| | PC₁ | | | | | | | (size >1 μm) |
| e 2.2 | PA₄/ | 2.01 | 1.03 | 9.30 | 1.01 | 1.01 | 0.98 | Flocculation |
| | PC₅ | | | | | | | (size >1 μm) |

The results clearly show that the nanoparticles obtained after mixing the polyelectrolytes in a ratio Z or a concentration C, not according to the invention, are larger than 500 nm, not according to the invention.

### Example 5

### Formulations According to the Invention Concentrated by Ultrafiltration

The formulation e 1.9 of Example 3 having a total polymer concentration of 1.65 mg/g is concentrated by a factor of approximately 8 by frontal ultrafiltration on a membrane having a cutoff of 10 kDa. The final polymer concentration obtained (measured by dry extract) is 13.4 mg/g. The size of the particles (average volume diameter) after concentration is 332 nm and the Zeta potential is −37 my.

This example therefore shows that it is possible to concentrate the obtained formulation by ultrafiltration without significantly changing the size and Zeta potential of the particles constituting this formulation.

### Example 6

### Formulations According to the Invention Incorporating Salmon Calcitonin (sCT) as Active Ingredient

The sCT is firstly mixed with the anionic polyelectrolyte PA and the PA/sCT complex thus obtained is subsequently mixed with the cationic polyelectrolyte PC.

More precisely, the anionic polyelectrolyte PA is diluted in a 10 mM solution of phosphate buffer and mixed with a solution containing 10 mg/g of sCT (Polypeptide Laboratories AB) so as to obtain a PA/sCT mixture having a concentration C₁ of anionic polyelectrolyte PA and a concentration Cₚ₁ of protein sCT. The mixture is stirred moderately for 1 h at ambient temperature.

A mass m₂ of cationic polyelectrolyte PC, diluted beforehand to concentration C₂, is added to a mass m₁ of the previous mixture PA/sCT maintained under moderate stirring.

The final mixture has a total polymer concentration C and a protein concentration Cₚ.

The concentration of active ingredient not combined with the polyelectrolytes is determined after separation by ultracentrifugation on ultrafilters having a cutoff of 30 kDa and assay of the filtrates by HPLC. In all cases it is strictly less than 5%.

The characteristics of the anionic and cationic polyelectrolytes used for this example are described in Examples 1 and 2.

TABLE 5

| | | | | Cₚ₁ | | | Cₚ | | | Volume | |
|---|---|---|---|---|---|---|---|---|---|---|---|
| | | m₁ | C₁ | (mg/g) | m₂ | C₂ | (mg/g) | C | | diameter | Zeta |
| Tests | Polymers | (g) | (mg/g) | (sCT) | (g) | (mg/g) | (sCT) | (mg/g) | Z | (nm) | (mV) |
| e 3.1 | PA₁/PC₃ | 14.97 | 1.03 | 0.10 | 1.95 | 7.98 | 0.09 | 1.83 | 0.49 | 118 | −46 |
| e 3.2 | PA₃/PC₃ | 4.99 | 1.00 | 0.49 | 10.02 | 1.00 | 0.16 | 1.00 | 0.48 | 136 | −35 |

The results show that the formulations according to the invention incorporating salmon calcitonin and polyelectrolytes according to the invention are composed of nanoparticles smaller than 500 nm.

### Example 7

### Formulation According to the Invention Incorporating Interferon α-2b (Ifnα) as Active Ingredient

The IFNα is firstly mixed with the anionic polyelectrolyte PA and the PA/IFNα complex thus obtained is subsequently mixed with the cationic polyelectrolyte PC. More precisely:

The anionic polyelectrolyte PA is diluted in a 10 mM solution of NaCl. A solution containing 2.3 mg/g of IFNα (Biosidus) is then added so as to obtain a PA/IFNα mixture having a concentration C₁ of anionic polyelectrolyte PA and a concentration Cₚ₁of IFNα protein. The mixture is maintained under moderate stirring for 14 h at ambient temperature.

A mass m₂ of cationic polyelectrolyte PC, diluted beforehand to the concentration C₂ in a 10 mM solution of NaCl, is added to a mass m₁ of the preceding PA/IFNα0 mixture maintained under stirring. The mixture is then stirred for 1 hour.

The final mixture has a total polymer concentration C and a protein concentration Cₚ.

The characteristics of the anionic and cationic polyelectrolytes used for this example are described in Examples 1 and 2.

TABLE 6

| | | | | Cₚ₁ | | | Cₚ | | | Volume | |
|---|---|---|---|---|---|---|---|---|---|---|---|
| | | m₁ | C₁ | (mg/g) | m₂ | C₂ | (mg/g) | C | | diameter | Zeta |
| Tests | Polymers | (g) | (mg/g) | (IFNα) | (g) | (mg/g) | (IFNα) | (mg/g) | Z | (nm) | (mV) |
| e 4.1 | PA₂/PC₁ | 9.47 | 1.04 | 0.27 | 15.50 | 1.16 | 0.10 | 1.11 | 0.74 | 174 | −46 |
| e 4.2 | PA₂/PC₂ | 8.97 | 0.89 | 0.30 | 17.05 | 1.38 | 0.10 | 1.21 | 0.70 | 194 | −43.5 |

The results show that the formulations according to the invention incorporating IFNα and polyelectrolytes according to the invention are composed of nanoparticles smaller than 200 nm.

1. Method for the preparation of nanoparticles with an average diameter less than or equal to 500 nm, comprising at least the stages consisting of:
(1) having an aqueous solution comprising nanoparticles of a first polyelectrolyte in the charged state bearing hydrophobic side groups;
(2) bringing said solution (1) together with at least one second polyelectrolyte of opposite polarity to that of the first polyelectrolyte, with the cationic polyelectrolyte being added to a solution of anionic polyelectrolyte in order to form a mixture with an excess of anionic charge; or the anionic polyelectrolyte being added to a solution of cationic polyelectrolyte in order to form a mixture with an excess of cationic charge; and
(3) having the nanoparticles thus formed;
with:
said anionic and cationic polyelectrolytes having a linear backbone of the polyamino acid type, devoid of side groups of the polyalkylene glycol type, and having a degree of polymerization less than or equal to 2,000;
the molar ratio Z of the number of cationic groups relative to the number of anionic groups in the mixture of the two polyelectrolytes being comprised between 0.1 and 0.75 or between 1.3 and 2; and
the total mass concentration C of polyelectrolytes being strictly less than 2 mg/g of said mixture.

2. Method according to the previous claim, characterized in that the molar ratio Z of the number of cationic groups relative to the number of anionic groups in the mixture of said anionic and cationic polyelectrolytes is comprised between 0.3 and 0.75, more particularly between 0.5 and 0.75, or between 1.3 and 1.5.

3. Method according to any one of the previous claims, characterized in that the total mass concentration C of polyelectrolytes in the mixture is comprised between 0.5 and 1.8 mg/g, in particular between 1 and 1.5 mg/g.

4. Method according to any one of the previous claims, characterized in that the mixture is produced at a pH ranging from 5 to 8, in particular from 6 to 7.5.

5. Method according to any one of the previous claims, characterized in that stage (2) comprises at least:
the preparation of an aqueous solution of the second polyelectrolyte, in particular with a pH value ranging from 5 to 8, and advantageously with a pH value identical to that of the aqueous solution of stage (1); and
the mixing of said aqueous solution of the second polyelectrolyte with said aqueous solution of stage (1).

6. Method according to any one of the previous claims, characterized in that the size of the nanoparticles varies from 20 to 300 nm, preferably from 50 to 200 nm.

7. Method according to any one of the previous claims, characterized in that said polyelectrolyte bearing hydrophobic side groups is capable of spontaneously forming nanoparticles when it is dispersed in an aqueous medium; with a pH ranging from 5 to 8, in particular water.

8. Method according to any one of the previous claims, characterized in that said anionic polyelectrolyte is of the following formula (I) or one of its pharmaceutically acceptable salts, 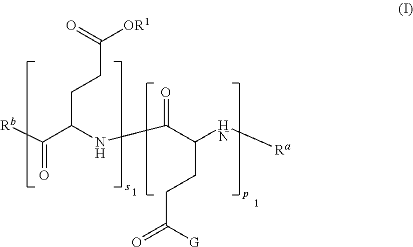
in which:
represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
R⁵ represents a hydrogen atom, a linear C₁ to C₁₀ alkyl group, a branched C₃ to C₁₀ alkyl group, or a benzyl group;
R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-, preferably alpha-tocopheryl-;
s₁ corresponds to the average number of non-grafted glutamate monomers, anionic at neutral pH,
p₁ corresponds to the average number of glutamate monomers bearing a hydrophobic group G,
p₁ optionally being zero,
the degree of polymerization DP₁=(s₁+p₁) is less than or equal to 2,000, in particular less than 700, more particularly ranging from 40 to 450, in particular from 40 to 250, and in particular from 40 to 150,
the chain formation of the monomers of said general formula (I) can be random, of monoblock or multiblock type.

9. Method according to any one of the previous claims, characterized in that said cationic polyelectrolyte is of the following formula (II) or one of its pharmaceutically acceptable salts, 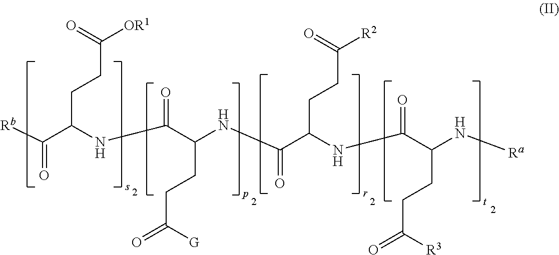
in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
R⁵ represents a hydrogen atom, a linear C₁ to C₁₀ alkyl group, a branched C₃ to C₁₀ alkyl group, or a benzyl group;
R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-, preferably alpha-tocopheryl-;
R² represents a cationic group, in particular arginine;
R³ represents a neutral group chosen from: hydroxyethylamino-, dihydroxypropylamino-;
s₂ corresponds to the average number of non-grafted glutamate monomers, anionic at neutral pH,
p₂ corresponds to the average number of glutamate monomers bearing a hydrophobic group G, and
r₂ corresponds to the average number of glutamate monomers bearing a cationic group R²,
t₂ corresponds to the average number of glutamate monomers bearing a neutral group R³,
s₂, p₂ and t₂ optionally being zero, and
the degree of polymerization DP₂=(s₂+p₂+r₂+t₂) is less than or equal to 2,000, in particular less than 700, more particularly varies from 40 to 450, in particular from 40 to 250, and in particular from 40 to 150,
the chain formation of the monomers of said general formula (II) can be random, of monoblock or multiblock type.

10. Method according to any one of the previous claims, characterized in that said nanoparticles of the first polyelectrolyte of the aqueous solution (1) are non-covalently combined with an active ingredient.

11. Method according to the previous claim, characterized in that said active ingredient is a molecule of therapeutic, cosmetic or prophylactic interest or of interest for imaging.
